(19) United States
(12) Patent Application Publication
(10) Pub. No.: US 2015/0330817 A1
(43) Pub. Date: Nov. 19, 2015

(54) Method and Device for Environmental and Health Monitoring

(71) Applicants: Sui Chun Law, Shaitin (HK); Paul Yiu Shum Ko, Shaitin (HK)

(72) Inventors: Sui Chun Law, Shaitin (HK); Paul Yiu Shum Ko, Shaitin (HK)

(21) Appl. No.: 14/808,937

(22) Filed: Jul. 24, 2015

Related U.S. Application Data
(63) Continuation-in-part of application No. 13/404,833, filed on Feb. 24, 2012, now Pat. No. 9,121,837.
Application No. 13/404,833 is a continuation-in-part of application No. 13/331,268, filed on Dec. 20, 2011, now abandoned.
Application No. 13/331,268 is a continuation-in-part of application No. 12/281,824, filed on Sep. 5, 2008, now Pat. No. 8,086,407.

(30) Foreign Application Priority Data
Mar. 10, 2006 (CN) 200610057261.2
Jul. 22, 2015 (HK) 15107004.2

(51) Int. Cl. G01D 21/02; G01N 33/00; G01W 1/00

(52) U.S. Cl. CPC G01D 21/02; G01W 1/00; G01N 33/0062; G01N 33/0032

(57) ABSTRACT

The present invention is related to an environmental monitoring and analyzing device. The device contains a plurality of sensors, a control unit and a display unit. The different types of sensor obtain values of different environmental parameters. The control unit receives the obtained values of the environmental parameters and compares the obtained values against predetermined standards and criteria which define parameter ranges of the different environmental parameters. A display unit displays a real-time air quality report comprising a user-friendly interpretation of the obtained values and recommendations in response to the obtained values. The present invention record and analyze the cause and consequence on the changed of level of different environmental parameters at different time duration, it also provide a tool to record and analyze the cause of some possible disease of the occupants.

### RELATED APPLICATIONS

This application claims priority to Hong Kong Application No. 15107004.2 filed Jul. 22, 2015, and is a continuation-in-part of U.S. application Ser. No. 13/404,833 filed Feb. 24, 2012, which application is a continuation-in-part of U.S. application Ser. No. 13/331,268 filed Dec. 20, 2011, which application is a continuation-in-part of application Ser. No. 12/281,824 Sep. 5, 2008, which is a national stage filing of PCT/CN2007/000736 filed Mar. 7, 2007, which application claims priority to Chinese Application No. 200610057261.2 filed Mar. 10, 2006, the entire content of each of which are incorporated herein by reference.

### TECHNICAL FIELD

The present invention is related to the technology of environmental monitoring. More specifically, it is related to a device and a method for environmental monitoring and analyzing.

### BACKGROUND

As the problems of indoor air pollution are getting severe, the public concerns on the conditions of their living and working environment as well as the health effects by the indoor air quality are increasing. At the same time, the guidelines and the rules to control and regulate the indoor air quality have been established in different countries. Hence, the demand for instruments and equipment on monitoring the air quality is increasing.

Conventionally, there are two major types of instruments for monitoring the indoor air quality. The first type of environmental monitoring instruments is employed mainly in the research laboratories. They are of considerably large scale. The second type belongs to the portable survey type instruments and they are much more compact in size.

The results obtained by the first type of environmental monitoring instrument are highly precise and accurate. Nevertheless, the prices of this type of instruments are significantly high. The operations of these instruments are complicated and only manageable by well trained and skillful technicians.

Generally speaking, each second type environmental monitoring instrument is equipped with a sensor for measuring a particular environmental parameter. The size of this type of instrument is therefore comparatively compact. Nevertheless, as different environmental parameters are inter-correlated, the level of a single parameter is usually affected by the levels of the other parameters. To obtain the level of a particular environmental parameter with a single sensor is usually not an all-round method. The precision obtained would be low. For examples, to measure the concentration of the volatile organic compounds solely by the photo-ionization detection method may give an inaccurate result as the detection method is easily affected by the temperature and relative humidity of the environment. In addition, different types of sensors with different working principles give different outcomes when they are employed for monitoring the same environmental parameter. For these reasons, there are usually difficulties to standardize the detection methods for the environmental parameters. In order to ensure an adequate and a moderately accurate result can be obtained for a single parameter, several instruments are usually brought to site during measurement. The results obtained are then evaluated together during analysis. The measurement processes by multiple instruments are rather inconvenience.

Nevertheless, for both types of environmental monitoring instruments being mentioned, only the raw data would be simply displayed and output. None of them would provide systematically analyzed information as described in the present invention.

### SUMMARY OF THE INVENTION

At least one advantage of the present invention is to address the deficiencies of current environmental monitoring devices and methods.

It has the further advantage to provide a device and a method for environmental monitoring and analyzing. Different levels of the environmental parameters are detected and measured at the same time. They are then judged and analyzed systematically.

It has the further advantage to provide a systematically analyzing and health assessment on the co-relationship between the different obtained environmental parameters and the health parameters and/or conditions of the occupants. The health assessment further includes the assessment and evaluation related to the possible disease and health risk on respiratory system of the occupants by the environmental parameter. Based on the result of the health assessment, the device and method further provides information and recommendations to improve health of the occupant,

The present invention of a method and device further provides a tool to record and analyze the cause and consequence on the changed of level of different environmental parameters at different time durations.

The present invention of a method and device further provides a tool to record and analyze the cause of some possible disease of the occupants; it also provides a tool to record and analyze the cause and source of irritating the respiratory system.

The present invention of a method and device further provides a forecast for the occupant, upon co-relating the demographic group of the occupant, on that the extended life expectancy or reduced life expectancy that he could expected from the changed of level of different environmental parameters.

The present invention of a method and device further provides a real time report, which is comprehensive to the user, including comment on the air quality, including at least one of the following information:
(1) Comment on the overall air quality;
(2) Comment and forecast on the levels of different environmental parameters which are obtained by the sensors of the environmental monitoring device;
(3) Comment and forecast on the levels of different environmental parameters which are not obtained by the sensors of the environmental monitoring device;
(4) A recommendation to the users or the occupants on how to improvement of the environment condition, basing on the obtained result;
(5) Comment on the health effect by the levels of different environmental parameters and/or the health effect of different pollutants sources which detected by the plurality of sensors;
(6) Comment and/or forecast on the overall improved or deteriorated health conditions on the change of different environmental parameters;
(7) Comment on the air exhausting conditions, air ventilation conditions, operation condition of air filtration device being employed in the environment;
(8) Comment on to the extent the values of different environmental parameter (which obtained by the plurality of sensors) being effected by the regional outdoor air quality;
(9) Comment on time dependent values between the indoor environment and outdoors environment, such as the heat exchange rate, the disperse rate, the pollutants emission rate, the pollutant removal rate, the air-change rate;
(10) Comment on the operation of at least one of the following equipment to improve the air quality at an energy efficiency way: air exhausting system, humidifier, dehumidifier, air warming device, air cooling device, air filtration device, combustion oven or device, ventilation fan, vacuum cleaner; and/or
(11) Comment on at least one of the following human action: to open the window, decrease the number of occupants, leave the place immediately, not to smoke, wear a mask, carry out disinfection and cleaning works, remove dust.

The construction of the device is simple. It is easily operated even by the non-technical users. The environmental parameters evaluated are highly accurate and precise.

The present invention provides a device to monitor the environment, and to solve the problems incurred by the conventional environmental monitoring instruments.

An environmental monitoring method and device, comprising:

a plurality of sensors of different types, where different types of sensor obtain values of different environmental parameters;
a control unit performs real-time analysis of different environmental parameters by considering the interrelationship of the obtained values of the different environmental parameters, providing an instant level assessment of at least one environmental parameter not obtained by the plurality of sensors.

The environmental monitoring device, wherein the control unit further comprises a central processing unit; the central processing unit stores predetermined standards and criteria; the predetermined standards and criteria which define the parameter ranges of the different environmental parameter.

The environmental monitoring device further comprise a display unit; the display unit displays a real-time air quality report; the real-time air quality report comprising the simultaneous forecast of the instant level assessment of the environmental parameter not obtained by the plurality of sensors.

The real-time air quality report further comprises a recommendation based on the obtained values.

The control unit further receives the obtained values of the environmental parameters and to compare the obtained values against predetermined standards and criteria which define parameter ranges of the different environmental parameters.

The predetermined standards and criteria which define the parameter ranges of the different environmental parameter includes the first judgment principle defines the parameter ranges for each measured environmental parameter;

The predetermined standards and criteria which define the parameter ranges of the different environmental parameter further includes the second judgment principle defines the conditional arrays; at least two parameter ranges defined by the first judgment principle are employed the parameter ranges for defining each conditional array.

The predetermined standards and criteria which define the parameter ranges of the different environmental parameter further includes the third judgment principle defines the categories for each measured environmental parameter.

The predetermined standards and criteria which define the parameter ranges of the different environmental parameter further includes a forth judgment principle defines an overall air quality level, which is defined by the air-quality-level judgment standards based on the combination of different categories of the measured environmental parameters.

The environmental monitoring device further comprises a memory to store the predetermined standard and criteria.

The real-time air quality report comprising a simultaneous forecast to provide an instant level assessment of at least one environmental parameter not obtained by the plurality of sensors further including at least one selected from the group consisting of: formaldehyde, airborne bacteria, radon and nitrogen monoxide, carbon dioxide, carbon monoxide, respirable suspended particulates, ozone, air flow rate, fungi level, total volatile organic compounds, temperature, relative humidity, dew point, air pressure, wind speed, overall air quality.

Based on the instant level assessment of (i) the environmental parameters obtained by the plurality of sensors, and/or (ii) the environmental parameters not obtained by the plurality of sensors and the information obtained from the health parameter sensor and/or monitor, the environmental monitoring device further provides a report to the occupant comprising any comments on: (a) potential health problems caused by environment, (b) health risk assessment, (c) recommendations on improving the environment, (d) improved health conditions of the occupant based on any improved environmental parameters.

The real-time air quality report also comprises a comment on any one of the following: the current and recommended operation conditions of the air exhausting conditions, air ventilation conditions, operation condition of air filtration device.

The real-time air quality report also comprises a comment on the health effects toward the occupant, which are caused by the different levels of the environmental parameters obtained by the plurality of the sensors, and/or by the instant levels assessment of different levels of environmental parameters which were not obtained by the plurality of sensors.

Based on the individual levels and the combinations of different levels of the environmental parameters obtained by the plurality of the sensors, and/or by the instant assessments of different levels of environmental parameters which were not obtained by the plurality of sensors, the real-time air quality report further comprises the comment on the health effect on the short term and the long term exposure by the environmental parameters.

The real-time air quality report further comprises comment on the sources which irritate the eyes and the respiration system, the detected and/or the recommended number of indoor occupant.

The environmental monitoring device further comprises a plurality of health parameter sensors of different types, where different types of health parameter sensor obtain values of different health parameters of at least one occupant and wherein, the occupant is someone who is living in, and/or being generally resides in, and/or performs his/her general activities in the location where the value of the different environmental parameters being obtained.

The health parameter sensors includes at least one selected from the group of sensor consisting of: heart pulse, heart rate, blood pressure, breathing rate, body movement sensor, sensor to detect the breathing patter of the occupants, the sensors to detects the occupant activities, stethoscope, body weight, blood oxygenation, and camera of different types.

The environmental monitoring device further being linked to a surveillance system, wherein the surveillance system comprises at least one surveillance device to monitor of the behavior, activities, or other changing information of the occupant(s); the surveillance devices including at least one selected from the group consisting of: computer, telephones, cameras, biometric device, Radio Frequency Identification (RFID) tagging devices, global positioning system device, mobile phone devices, human microchips device.

The environmental monitoring device further being linked to at least one environmental monitoring station, databases, information center and/or system regarding the pollutant levels and/or the pollution index of the regional outdoor air quality; the regional outdoor air quality further comprises the pollutants level(s) and/or pollution index(es) of environmental parameters which are of concerned.

The level(s) and/or index(es) of the environmental parameters of the regional outdoor air quality which are of concerned are compared with the values of different environmental parameter which obtained by the plurality of sensors. Upon comparison, comments on to the extent the values of different environmental parameter (which obtained by the plurality of sensors) being effected by:

(a) the regional outdoor air quality; and/or
(b) pollutants level(s) and/or pollution index(es) of environmental parameters were being concluded and being further addresses in the real-time air quality report. The comments further comprise any one of on time dependent values between the indoor environment and outdoors environment, such as the heat exchange rate, the disperse rate, the pollutants emission rate, the pollutant removal rate, the air-change rate.

The real-time analysis of the obtained values of the different environmental parameters is performed by considering the interrelationship of the obtained values of the different environmental parameters in order to interpret the obtained values and make the recommendation based on the obtained values.

The recommendation further includes the comment on the operation of at least one of the following equipment: air exhausting system, humidifier, dehumidifier, air warming device, air cooling device, air filtration device, combustion oven or device, ventilation fan, vacuum cleaner.

The recommendation further includes the comment on at least one of the following human actions: to open the window; decrease the number of occupants; leave the place immediately; not to smoke; wear a mask; carry out disinfection and cleaning works; and remove dust.

At least one timer counter is included in the device for monitoring the measurement period of the plurality of sensors. The average levels of different environmental parameters by the plurality of sensors under their respective measurement periods are obtained.

The device enables the user to setup the measurement period and measurement interval for each sensor.

The device also enables the user to setup the operation condition as the operation criteria for the sensor, which is based on the obtained values of the environmental parameters of the other sensor.

For example, when the carbon dioxide of a heated metal oxide-type sensor is in use, pre-heating of the sensor for first 5 minutes is usually required; the values obtained in the first 5 minutes during the pre-heating status are not accurate and will not be counted for the average level of the carbon dioxide.

For example, when the radon level is to be obtained by the radon sensor, the relative humidity should be set below 40% for an accurate result to be obtained. The comment of setting relative humidity below 40% is an operation criterion for setting as the operation of the radon sensor.

The real-time air quality report comprises a simultaneous forecast based on the said average levels of the different environmental parameters to provide an instant average level assessment of at least one environmental parameter not obtained by the plurality of sensors.

The device also enables a user to preset the calculation and rule out criteria which is to eliminate any unexpected, abnormal, out of the standard deviation measurements of the obtained average levels of different environmental parameters by different types of sensor at any instant.

The said preset calculation and rule out criteria help to prevent inaccuracies and errors which may be introduced to the calculation during the obtaining of the average levels of the environmental parameters, which may then result in a misleading assessment and forecasting of the environmental parameters which are not obtained by the plurality of sensors.

By the application of the preset the calculation and rule out criteria, the error caused by sudden change of the values of certain environmental parameters by any unexpected disturbance can be ignored.

For example, when a woman with perfume walks by the formaldehyde sensor or total volatile organic compound sensor which are used for measuring the concentration of formaldehyde and total volatile organic compound in the environment, the sudden rise in the formaldehyde and total volatile organic compound can be ruled-out and be ignored based on the preset calculation and rule out criteria. These unexpected rises of the sensor readings at a particular instant will not be counted when computing the average levels of the environmental parameters. The misleading assessment and forecasting results due to the instant rising of the values of the formaldehyde and total volatile organic compound be ignored.

During the setup of the preset calculation and rule out criteria, the user can decide the number of samplings per sensor and the sampling time per sensor in each measurement period. The user can also decide the maximum and minimum numbers of sampling values which are to be ruled out during the calculation of the average level of the environmental parameters. The user can employ a normal average calculation for each particular period of measurement or employ a rolling average calculation for the long term operation of the device.

The said setup preset calculation and rule out criteria and the setup of the said measurement period and measurement interval for each sensor can be done by user any time before or during the operation of the device. The user can input and store the setup into the control unit of the device. The input method can be made by direct key-in through the input port or synchronized by a computer or flash memory (as indicated in the FIG. 1)

The air flow rate, heat conduction rate, disperse rate of the pollutants, pollutants emission rate, pollutant removal rate, air-change rate, and other time dependent values can further be assessed and forecasted by the device by considering:

(1) the interrelationship of instant and/or the average level of the obtained environmental parameters; or

(2) the interrelationship of said levels of the environmental parameters which are not obtained by the plurality of sensors; or

(3) The interrelationship of (1) and (2) of the above against the timing factor.

Mathematic calculations by mean of calculus and logarithms, such as integration or differentiation, log and antilog may be applied as the equations and the rules of calculations to dictate for the time dependent values of the air flow rate, the heat conduction rate, the disperse rate of the pollutants, the pollutants emission rate, the pollutant removal rate, the air-change rate, and other time dependent values. The rules of calculations shall be preset in the control unit of the device.

For example, in the assessment of the time dependent values, the decay rate of one environmental parameter (the pollutants) obtained by one sensor, the following equation is applied:

Cₜᵢ=Cᵢe^{-kti}

Cₜᵢ is the value of the environmental parameters (e.g. the pollutant concentration) at the time ti, •g/m³

Cᵢ is the initial value of the environmental parameters (e.g. pollutant concentration) at ti=0, ••g/m³
k is the decay constant, hr⁻¹
tᵢ is time, hr

The decay constant, k, is obtained by using the linear regression on the (ln Cᵢ−ln Cₜᵢ) and tᵢ using the formula:

$$k = \frac{Σ {t}_{i} ( ln {C}_{i} - ln {C}_{{t}_{i}} ) - \frac{Σ {t}_{i} Σ ( ln {C}_{i} - ln {C}_{{t}_{i}} )}{n}}{Σ {t}_{i}^{2} - \frac{{( Σ {t}_{i} )}^{2}}{n}} {hr}^{- 1}$$

where:

n=number of data points

The decay rate can be calculated by

Decay rate=Ef×k

where:

Ef=a constant of environmental factor

The value of Ef is also an environmental factor of at least one of the following parameters/index(es)/level(s):
(1) the regional outdoor air quality; and/or
(2) pollutants level(s) and/or pollution index(es) of environmental parameters;
(3) temperature of the environment;
(4) temperature of the outdoor environment; and
(5) activities of the occupants within the indoor environment where the pollutant concentration (i.e., the values of different environmental parameters being obtained by the different types of sensors, or the values of the environmental parameters being simultaneous forecast, and not obtained by the plurality of sensors).

Thus, the air flow rate, heat conduction rate, disperse rate of the pollutants, pollutants emission rate, pollutant removal rate, air-change rate, and other time dependent values can further be assessed and forecasted by any one of the following factors: the regional outdoor air quality; the pollutants level(s) and/or pollution index(es) of environmental parameters, the temperature of the environment; temperature of the outdoor environment; activities of the occupants within the indoor environment where the pollutant concentration (i.e., the values of different environmental parameters) being obtained by the different types of sensors, or the values of the environmental parameters being simultaneous forecast, and not obtained by the plurality of sensors)

The decay rate of the value of the environmental parameters (e.g. the pollutants) can be interpreted in the following manner. As decay rate of the value of the environmental parameters (e.g., the pollutants) is equal to the removal rate of the environmental parameters (e.g. the pollutants) minus the emission/generating rate of the environmental parameters (e.g. the pollutants). A positive decay rate indicates the removal rate of the environmental parameters (e.g. the pollutants) is greater than the emission/generating rate of the environmental parameters (e.g. the pollutants), whereas a negative result indicates that the emission/generating rate of the environmental parameters (e.g. the pollutants) is greater than the removal rate of the environmental parameters (e.g. the pollutants). A zero value indicates an equilibrium condition has reached where the removal rate of the environmental parameters (e.g. the pollutants) is equal to the emission/generating rate of the environmental parameters (e.g. the pollutants).

By interpreting the time dependent values of different environmental parameters obtained by the plurality of sensors, the following interpretation can be assessed and forecast:

(1) The other time dependent values of the environmental parameter which obtained by the plurality of sensors
(2) The time dependent values of at least one environmental parameter not obtained by the plurality of sensors.

For example, in obtaining the time dependent value, the decay rate of the carbon dioxide which is obtained by the carbon dioxide sensor, the emission/generating rate by the number of occupants, and the removal rate of carbon dioxide by ventilation between the indoor and outdoor can be assessed and forecast. For example, a positive decay rate of the carbon dioxide indicated that the removal rate of carbon dioxide by ventilation to the outdoor environment is higher. For example, a zero decay rate of carbon dioxide means the removal rate of carbon dioxide by ventilation is just good enough to cater the emission/generation rate by the number of occupants. In this way, the other time dependent values (the removal rate of carbon dioxide and the emission/generation rate of the carbon dioxide in this example) of the environmental parameter which obtained by the plurality of sensors can be assessed and forecasted.

For example, in an enclosed room where a zero decay rate of carbon dioxide is obtained. The result indicated that there is no emission/generating carbon dioxide as well as the removal of carbon dioxide by the ventilation is happened. The result also indicates that the other environmental pollutants such as respirable suspended particulate, formaldehyde, volatile organic compound which are generated by the ventilation means will become insignificant or even zero concentration too. Thus, in that case, if a negative value of the decay rate of the respirable suspended particulate is obtained at that moment, and if no other respirable suspended particulate removal equipment (e.g., air purifier) is in used, the emission/generation rate shall be equal to the absolute value of that negative value decay rate. Thus, by the interpretation of the interrelationship of instant and/or the average level of the obtained environmental parameters by a plurality of sensors, other time dependent values of the environmental parameter which obtained by the plurality of sensors can be assessed and forecasted.

By considering the time dependent values of some environmental parameter which the values are obtained by the plurality of sensors, the time dependent values of some environmental parameters not obtained by the plurality of sensors (e.g, airborne bacteria level, total volatile organic compounds, fungi, etc. can be assessed and forecasted. For example, a negative or zero decay rate of carbon dioxide with a negative decay rate of respirable suspended particulate can forecasts and tells there is a positive growing of airborne bacteria level due to (1) the poor ventilation and (2) positive generation/emission of respirable suspended particulate which serves as the nutrient source for airborne bacteria. For example, a positive decay rate of carbon dioxide with a negative decay rate of volatile organic compounds can tells a very bad condition that the emission/generation rate of volatile organic compounds is too high over ventilation. The concentration of volatile organic compound is keep increasing and become too significant even within good ventilation room, the emission/generation rate of the formaldehyde can therefore forecasted to be a very high level.

At least one communication input and output port (as indicated in the FIG. 1) is included in the device. When a plurality of devices of the same type are connected together with the said communication input and output port, the trends of any of the air flow path, heat conduction path, disperse path of the pollutants, pollutants emission path, pollutant removal path, air-change path is forecast and can be displayed in the said a real-time air quality report.

The environmental monitoring device is further connected to at least one health parameter sensor and/or monitor by means of wiring or wireless data communication. The health parameter sensor and/or monitor obtain the information on the health condition of at least one occupant who resides at the location where the value of different environmental parameters being obtained by the plurality of sensors.

The environmental monitoring device further performs the real-time analysis by considering the interrelationship of the obtained values of the different environmental parameters and the data obtained by the health parameter sensor and/or monitor.

The environmental monitoring device further comprises input/output ports for transferring information to other devices through an infra-red interface, or a Bluetooth interface or other wireless interface.

The environmental monitoring device is further linked to a cloud storage system and/or a cloud computing system through a wired or wireless data transmission method.

The environmental monitoring device, wherein the obtained values of the different environmental parameters are dynamically uploaded and stored in a cloud storage system through a wired or wireless data transmission method.

The cloud storage system is simultaneously linked to multiple units of environmental monitoring device through a wired or wireless data transmission method. The values of the environmental parameters obtained by the plurality of sensors from multiple units of environmental monitor device are dynamically uploaded and co-stored to the cloud storage system.

The environmental monitoring device, wherein the real-time analysis of different environmental parameters is performed in the cloud computing system; the predetermined standard and criteria are stored in the memory of the cloud storage system; the stored predetermined standard and criteria are extracted from the cloud storage system to the cloud computing system for performing the real-time analysis of different environmental parameters.

The real time analysis of different environmental parameters includes of the real time analysis by the cloud computing system on the values of different environmental parameters which obtained from the multiple units of environmental monitoring device. In other words, the real time analysis is performed on the environmental parameters obtained from the plurality sensors of the multiple units of environmental monitoring device.

The device also includes a power saving function, which can work together with the power management components of the device.

The environmental monitoring device, wherein the plurality of sensors and the control unit are powered by different power sources, (the plurality of sensors and the control unit are in different casings and in separate units), the obtained values of the different environmental parameters by the plurality of sensors are transmitted to the control unit through a wiring or a wireless data transmission method.

The environmental monitoring device, wherein the control unit and the central processing unit are powered by different power sources, (the control unit and the central processing unit are in different casings and in separate units), the obtained values of the different environmental parameters by the plurality of sensors are transmitted to the control unit through a wired or a wireless data transmission method.

The environmental monitoring device, wherein the control unit and the display unit are powered by different power sources, the display unit and the control unit are in different casings and in separate different units, and the control unit transmits the real-time air quality reports to the display unit for display through a wired or a wireless data transmission method.

The environmental monitoring device, wherein each individual sensor or several sensors from the plurality of sensors are powered by different power sources. Each individual sensor or several sensors of the plurality of sensors are in different casings and in separate different units relative to each other.

The environmental monitoring device, wherein the control unit is further linked by a wired or a wireless data transmission method to a single or a plurality of power control(s), where the power control(s) are for supplying the power to operate different environmental device(s). When the environmental device (s) are or are not operating, the values of different environmental parameters obtained by the plurality of sensors will be different.

The environmental monitoring device, wherein the control unit is further linked by a wired or a wireless data transmission method to a single or a plurality of environmental device(s). When the environmental device (s) is/are in operating or is/are stop operating, the values of different environmental parameters obtained by the plurality of sensors will be different.

The environmental monitoring device wherein the cloud storage system and/or cloud computing system is further linked by a wired or wireless data transmission method to a single or a plurality of environmental device(s) which the when the environmental device (s) is/are in operating or is/are stop operating, the values of different environmental parameters obtained by the plurality of sensors will be different.

The environmental monitoring, wherein the recommendation include the recommendation of the operation status of the environmental device(s).

The environmental monitoring device, wherein the control unit further controls the operation of the environmental device(s) according to any one of the following (a) the recommendation; (b) the according to the instant level assessment of at least one environmental parameter not obtained by the plurality of sensors.

The environmental monitoring device a, wherein the predetermined standards and criteria includes a first judgment principle, where the first judgment principle defining at least two parameter ranges for each environmental parameter, and a corresponding recommendation for each of the at least two parameter ranges.

The environmental monitoring device, wherein the predetermined standards and criteria includes a second judgment principle, where the second judgment principle defines at least one conditional array, and the at least two parameter ranges defined by the first judgment principle are used as the parameter ranges for defining each conditional array, and a message corresponding to potential problems for each of the at least one conditional array is provided.

The environmental monitoring device, wherein the predetermined standards and criteria includes a third judgment principle, where the third judgment principle defines at least two categories for each environmental parameter and air-quality-level judgment standards for air quality levels are defined based on a combination of different categories of the obtained environmental parameters, and a message corresponding to air quality level by the air-quality-level judgment standards is provided.

The environmental monitoring device, wherein the different environmental parameters are selected from a group consisting of: temperature, relative humidity, volatile organic compounds, carbon monoxide, carbon dioxide, dust, ozone, carbon dioxide, air flow rate, radon, and formaldehyde, number of occupants, noise, light.

The environmental monitoring device, wherein the predetermined standard and criteria which define parameter ranges of different environmental parameters will be modified dynamically; the modified predetermined standard and criteria will be uploaded to the control unit by a wiring or wireless data transmission method.

The environmental monitoring device, wherein the predetermined standard and criteria which define parameter ranges of different environmental parameters will be modified dynamically based on the operation of the environmental device.

When the parameter ranges of first judgment principle of the conditional array reach to the preset values, the parameter ranges of another first judgment principle which was originally consider by same said conditional array will become suppressed, hidden, ignored, and not being considered in the assessment and forecast of the environmental parameter that is not obtained by the plurality of sensors. The original conditional array will automatically shift to another conditional array. For examples, in one the conditional array, the environmental parameters of temperature and total volatile organic compound are employed for assessment of the level of formaldehyde. When the temperature is within the range of 25.5° C. to <35° C. (which is the optimal range for emission of the formaldehyde), and when the level of total volatile organic compound is 600 ••g/m³ above, the formaldehyde level is forecasted to be a problematic and messages of this potential problem will be displayed. However, when the level of total volatile organic compound is in the range of 3000 to <25000 •g/m³, the reading from the temperature will become ignored in the assessment and forecast of the level of the formaldehyde. This is because the level of the total volatile organic compound is already dominant over the temperature in the assessment and forecasting of the level of formaldehyde. In indoor environment where the concentration of total volatile organic compound is in the range of 3000 to <25000 •g/m³, the concentration of formaldehyde is already displayed in an alert level regardless the temperature of the environment.

When the parameter ranges of said another first judgment principle which was originally considered by the same said conditional array become suppressed and ignored. The sensor for obtained the parameter ranges of said another first judgment principle will be turned off automatically and temporarily for power saving. The sensor will become re-activated and the parameter ranges of the respective environmental parameter will become re-considered again at the time the parameter ranges of first judgment principle of the conditional array returned and fell back to original defined ranges.

This including this power saving function is especially beneficial for some sensors with required huge power consumptions, or sensors that needed to work with heating elements. The function helps to prevent the decay of the power source when battery or re-chargeable battery is in used, which would other affect the functioning of the device. It can help to prevent the generation of unwanted heat source or wasted heat which would affect the functions of some other sensor. It can help provide a stable and sustainable power source for all sensors of the device.

Besides, the device further comprising a recommendation to address the potential problems.

The device further comprising an at least one input/output port and it is being connected to a central processing unit of at least one air treatment unit; the central processing units of the said air treatment unit receive the messages corresponding to the said real-time air quality report from the device; and based on the message to establish setting and parameter values for the operating condition of the said air treatment unit.

The said air treatment includes any unit and modules of the air equipment containing one or the combination of the components from: fan of any type, blower, pump, drawer, filtration apparatus and/or filter for air pollutants of any type, apparatus for sterilizing the air, apparatus for environmental humidity controlling, apparatus for the environmental temperature controlling, apparatus for environmental air flow controlling, apparatus for controlling environmental brightness.

The setting and parameter values for the operating condition of the said air treatment unit based on the messages corresponding to the said real-time air quality report at least one or the combination of the following:

the operating time, air flow rate, air flow path, the on and off of the air treatment unit; the on and off, the temperature setting of the apparatus for environmental humidity controlling;

the on and off of and the temperature setting of the apparatus for environmental temperature controlling;

the on and off, and power setting for the apparatus for sterilizing the air.

In another embodiment, the device is a part of the component which is being included in any unit and modules of the air equipment containing one or the combination of the components from: fan of any type, blower, pump, drawer, filtration apparatus and/or filter for air pollutants of any type, apparatus for sterilizing the air, apparatus for environmental humidity controlling, apparatus for the environmental temperature controlling, apparatus for environmental air flow controlling, apparatus for controlling environmental brightness. The control unit of the device establishes the setting and the parameter values for the operating condition of the air equipment based on the obtained values of the environmental parameters and/or the simultaneous forecast and instant level assessment of at least one environmental parameter not obtained by the plurality of sensors. In such case, the control unit of the device is included in to the central processing unit of the air equipment.

The control unit of the device comprises:

a power supply;

control circuit;

input circuits;

output circuit;

a central processing unit; and

a memory to store the predetermined standards and criteria for judging the environmental parameters, messages corresponding to interpretations, recommendations and potential problems of the parameter ranges;

the power supply and control circuit connecting an external power supply to the device;

the input circuit collecting the obtained values from the sensors and outputting them to the central processing unit;

the central processing unit analyzing the obtained values based on the predetermined standards and criteria and defining the parameter ranges of each environmental parameter, and to output the interpretation and recommendation of each parameter range for display by the display unit.

The input circuit includes an analog to digital converter and a low pulse timer.

The present invention also offers a method to monitor and analyze the environment, comprising:

obtaining values of environmental parameters;

comparing the obtained values of the environmental parameters against predetermined standards and criteria which define parameter ranges of the different environmental parameters in a control unit; and

displaying a real-time air quality report from a control unit comprising a simultaneous forecast to provide an instant level assessment of at least one environmental parameter not obtained by the plurality of sensors.

wherein real-time analysis of the obtained values of the different environmental parameters is performed by considering the interrelationship of the obtained values of the different environmental parameters in order to interpret the obtained values and make recommendations based on the obtained values.

The real-time air quality report comprising a comment on at least one of the following: the conditions of the air exhausting conditions, the operation condition of the air filtration device, the sources which irritate the eyes and the respiration system, the number of the of indoor occupant.

The real-time air quality report further comprising messages corresponding to interpretations, recommendations and potential problems of the parameter ranges.

The real-time air quality report further comprising a user-friendly interpretation of the obtained values based on the parameter ranges.

The real-time air quality report further comprising a recommendation in response to the obtained values based on the parameter ranges that is easily understood by a non-technical user is included.

The said recommendation further includes the comment on
(i) the operation of at least one of the following equipment: air exhausting system, humidifier, dehumidifier, air warming device, air cooling device, air filtration device, combustion oven or device, ventilation fan, vacuum cleaner; and/or
(ii) at least one of the following human actions: to open the window, decrease the number of occupants, leave the place immediately, not to smoke, wear a mask, carry out disinfection and cleaning works, remove dust.

The measurement period of the plurality of sensors are monitored by at least one timer counter. The average levels of different environmental parameters by the plurality of sensors under their respective measurement periods are obtained. The real-time air quality report comprising a simultaneous forecast based on the said average levels of the different environmental parameters to provide an instant average level assessment of at least one environmental parameter not obtained by the plurality of sensors is displayed.

The air flow rate, heat conduction rate, disperse rate of the pollutants, pollutants emission rate, pollutant removal rate, air-change rate, can further be assessed and forecasted by considering:
(1) the interrelationship of instant and/or the average level of the obtained environmental parameters; or
(2) the interrelationship of said levels of the environmental parameters which are not obtained by the plurality of sensors; or
(3) The interrelationship of (1) and (2) of the above;
   against the timing factor.

At least one communication input and output port is used for connecting the device of the same type which employing the said environmental monitoring method together, the trends of any of the air flow path, heat conduction path, disperse path of the pollutants, pollutants emission path, pollutant removal path, air-change path is forecasted and be displayed in the said a real-time air quality report.

The method enable the user to preset calculation and rule out criteria which is aim to eliminate any the unexpected, abnormal, out of the standard deviation of the obtained average levels of different environmental parameters by different types of sensor at any instant.

The predetermined standards and criteria of the mentioned method includes a first, second and third judgment principle,

the first judgment principle defining parameter ranges for the environmental parameters, corresponding recommendations for each parameter range are provided;

the second judgment principle defining conditional arrays, and at least two parameter ranges defined by the first judgment principle for use as parameter ranges for defining each conditional array, a message corresponding to potential problems and recommendations to address the potential problems for each conditional array are provided;

the third judgment principle defining at least two categories for each environmental parameter, and air-quality-level judgment standards for air quality levels are defined based on the combination of different categories of the obtained values, and

a message corresponding to air quality level by the air-quality-level judgment standards is provided.

The environmental parameter is any one from the group consisting of: temperature, relative humidity, volatile organic compounds, carbon monoxide, carbon dioxide, dust, ozone, carbon dioxide, air flow rate, radon, and formaldehyde.

When the parameter ranges of first judgment principle of the conditional array reach to preset values, the parameter ranges of another first judgment principle which was originally consider by same said conditional array will become suppressed and ignored. The original conditional array will automatically shift to another conditional array.

When the parameter ranges of said another first judgment principle which was originally considered by same said conditional array become suppressed and ignored. The sensor for obtained the parameter ranges of said another first judgment principle will be turned off automatically for power saving. The sensor will become re-activated when the values of the parameter ranges being re-considered again at the time the parameter ranges of first judgment principle of the conditional array returned to original defined ranges.

In the present invention, the values of different environmental parameters are obtained by different sensors. Real-time analysis of the obtained values of the different environmental parameters is performed by considering the interrelationship of the obtained values of the different environmental parameters.

A real-time air quality report is provided. The real-time air quality report comprising a user-friendly interpretation of the obtained values and a recommendation in response to the obtained values that is easily understood by a non-technical user. (In other words, the report includes the message corresponding to the potential problems based on the parameter ranges, the recommendations to address the potential problems and the message corresponding to the air quality level.) For certain environmental parameters, such as airborne bacteria and fungi, which need longer testing time by conventional methods They need hours for incubations by the conventional methods, the present invention would be able to provide an instant level assessment by means of forecasting, based on the (interrelationship/correlation) between different measured environmental parameters. For instance, in a warm and humid environment where the dust level has reached a certain high level (in an environment where the level of respirable suspended particulates is high), the pre-requisite conditions for growing and incubating the airborne bacteria are actually created. Based on the values of the temperature, relative humidity and level of respirable suspended particulates, the level of airborne bacteria can then be forecasted simultaneously. On another example, in an environment where the concentration of the carbon dioxide is sustained at high level, poor ventilation or too many occupants are implied. With the present invention, a user-friendly interpretation of the obtained value of the environment would be generated. The user-friendly interpretation could be the messages of recommendations such as “turn on the air exhausting system”, “decrease the number of occupants”, “open the windows” etc. The device by the present invention is structurally simple and low cost. The device can be handled by non-technical users easily.

The present invention further provides an environmental monitoring device and system to function as a personalized portable environmental monitoring device, the environmental monitoring device and system comprises at least one sensor module which contains at least one sensor to obtain values of at least one of the environmental parameters.

The sensor module obtains the values of the environmental parameter continuously. The sensor module may further obtain the values of the environmental parameters continuously at a pre-set time period by the occupant.

One sensor module or a plurality of sensor modules of the same or different types are connected to at least one device containing a localized central processing unit (LCPU device) through a wired or wireless data transmission method. A first level of environmental monitoring system is built by connecting the plurality of sensor modules together with at least one LCPU device.

If the sensor module(s) is/are connected to the LCPU device through a wired data transmission method, the sensor module(s) or LCPU device are embedded into same casing and supported to operate by the same power supply source(s).

If the sensor module(s) is/are connected to the LCPU device through a wireless data transmission method, the sensors modules and/or LCPU device can be in different casings and supported to operate by different power supply sources.

In any case, the sensor module(s) and/or the LCPU device may further be equipped with suitable casing and power supply, such that it can be clipped to the clothing and/or be carried by at least one occupant, and/or be put in any location as desired by the occupants temporarily or permanently.

At least one LCPU device is further connected to another system device containing a central processing unit (SCPU device) through a wired or wireless data transmission method. A second level of environmental monitoring system is built by connecting the plurality of LCPU devices together with at least one SCPU device.

A higher level of environmental monitoring system is built by connecting the plurality of SCPU device together with a higher-level system device containing a central processing unit (HCPU device) through a wired or wireless data transmission method. An even higher level environmental monitoring system can be further built by the same and/or similar infrastructure whenever necessary.

The environmental monitoring device and system further comprises at least one display unit. The display unit is integrated into the sensor module or is integrated to the LCPU device(s), the SCPU device(s), and/or the HCPU device(s).

At least one fundamental data related to the occupant personal information and/or the related to the environmental information is input to the LCPU device through an input port. The input port may be integrated to the sensor module, the LCPU device, the SCPU device(s), and/or the HCPU device(s).

The fundamental data comprises at least one personal data of the occupant and/or at least one data related to the occupant's environment. The fundamental data contain data which belongs to a non-time relating type of data and/or a time-relating type of data.

The fundamental data related to the occupant personal information comprises at least one of the following information of the occupant: gender, occupation, age, and/or health history. Other designated users or occupants can read the processed and/or un-processed fundamental data.

The fundamental data related to the environmental information comprises at least one of the following information: nature of the location (indoor/outdoor), function of the location, ventilation information (window open or closed, fresh air supplied available or not) in the location, furniture types in the location, air conditioning system (central air condition unit/window type air conditioner/split type conditioner) in the location, number of occupants, whether pet/plants are present in the locations, etc.

Each sensor module has its own unique identity to match with the obtained values of the environmental parameter(s) and the fundamental data in the whole environmental monitoring device and system.

Each LCPU device has its own unique identity to be identified by the SCPU device in the second level of environmental monitoring system.

Each SCPU device has its own unique identity to be identified by the HCPU device in the high level of environmental monitoring system.

When a one or a certain number of sensor modules within the first level of environmental monitoring system become disconnected from one LCPU device, or an LCPU device becomes disconnected to the sensors modules, the time and duration of disconnection is be recorded within the sensors modules and the LCPU device.

When one or certain sensor modules within the first within the detection range of the LCPU device and becomes connected with the LCPU device, the time and duration of the connection is recorded within the sensor modules and the LCPU device.

The time of connection and disconnection of the sensors modules with the LCPU device(s) is treated as one of the fundamental data which is related to the occupant's mobility.

At least one environmental equipment and operative device is further connected to the first level environmental monitoring system through a wired or wireless data transmission method.

The environmental equipment and operative device is employed for improving the indoor and/or outdoor air quality and/or regulate the thermal comfort parameter for the occupant.

The environmental equipment and operative device is an equipment or device for eliminating the disaster causes and/or sources.

At least one health care device and/or at least one medical device is further connected to the first level environmental monitoring system through a wired or wireless data transmission method.

At least one emergency device is further connected to the first level environmental monitoring system, through a wired or wireless data transmission method. The emergency device is employed for providing immediate action to eliminate, to reduce, to alert the occupant any endangered sources and causes.

The first level environmental monitoring system performs (a) at least an assessment and/or (b) at least a forecast, related to a health concern of the occupants and/or safety concern of the environment based on the values of the environmental parameters and the fundamental data, the result of the assessment is reported to the user through the display unit and the result of the assessment will further be employed to provide a command to control of any of the environment equipment, the operative device, the emergency device, a health care device, and/or a medical device.

The LCPU device further performs the assessment in related to the health concern of the occupants based on at least a judgment principle in relating to the personal data and the obtained values of the environmental parameter.

The LCPU device further performs the forecast in related to the health concern of the occupants, based on at least one time-relating type of the personal data and the obtained values of the environmental parameter.

The LCPU device further performs the forecast in related to the health concern of the occupants, based on at least one time-relating type of the personal data and the changing profile of the obtained values of the environmental parameter.

The LCPU device further performs the assessment related to the safety concern of the environment, based on at least one second judgment principle related to at least one fundamental data and the obtained values of the environmental parameter.

The LCPU device further performs the assessment in related to the safety concern of the environment, based on at least one non-time relating type of at least one fundamental data and the changing profile of the obtained values of the environmental parameter.

The LCPU device further performs the forecast related to the safety concern of the environment, based on at least one time-relating type of at least one fundamental data and the changing profile of the obtained values of the environmental parameter.

The LCPU device further performs the forecast related to the safety concern of the environment, based on at least one third judgment principle on the changing profile of the obtained values of at least two the environmental parameters.

The result of the assessment and/or forecast is further sent to another designated LCPU device within or outside the same first level environmental monitoring system, to inform, report to, and/or alert to other designated users/occupants. The results of the assessment are sent by the routing as: (1) the result of the assessment is first sent to the SCPU device at the upper level of the environmental monitoring system from the LCPU device, (2) from the SCPU device, the result of the assessment is sent to another LCPU device.

The result of the assessment and/or the forecast are further sent as feedback to the LCPU and/or another designated LCPU device, within or outside the same first level environmental monitoring system, and may be employed to update any judgment principle for performing the future assessment.

The result of the assessment and forecast related to the health concern of the each individual occupant and/or safety concern of the environment performed by the LCPU device is sent to the SCPU device.

The obtained values of environmental parameter(s) together with the fundamental data undergo a first level of data processing by the LCPU device to generate some summarized information. The summarized information is then sent to the SCPU device.

The data processing of the obtained values of environmental parameter(s) and/or the summarized information of them includes at least one of the following: an average, a rolling average, a median, mean, an value on the confident interval at specified percentage of the obtained values; the increasing/decreasing rate of the obtained values, and the frequency and duration of when the alerted values is/are reached;

The data processing of the fundamental data and/or the summarized information includes classifying the fundamental data into different groups/batches by the LCPU device.

The SCPU device is further connected to at least one environmental equipment and operative device, through a wired or wireless data transmission method. The environmental equipment and operative device is employed for improving the indoor and/or outdoor air quality and/or the thermal comfort of the occupant.

The second level environmental monitoring system is further connected to at least one emergency device, through a wired or wireless data transmission method. The emergency device is employed for providing immediate action to eliminate, reduce, and/or alert the occupant of any endangered sources and causes.

The second level environmental monitoring system is further connected to at least one external database and obtains the values of at least one external parameter from the external database(s). The said external parameter is one factor directly/indirectly affecting the obtained values of the environmental parameters. (e.g., outdoor Air Index).

The values of the external parameter will be employed to adjust the judgment principle, which being employed by the LCPU device to perform the assessment in related to the health concern of the occupants.

To save the computing resources of the SCPU device and allow a quick instruction to be provided by the SCPU to any equipment and device and/or to any LCPU device connecting to it directly or indirectly, a data processing is carried out by the LCPU device, on the obtained values of the environmental parameter(s) and the fundamental data within the first level of environmental monitoring system.

In order to reduce the burden of the SCPU device, the processed data is sent to the SCPU device from the LCPU device, and a further data processing, a second level data processing will be carried out whenever necessary.

The second level data processing comprises computing the processed data from the LCPU device to obtain at least one of the following: an average, a rolling average, a median, mean, an value on the confident interval at specified percentage of the processed data; the increasing/decreasing rate of the processed data; and the frequency and duration of when the alerted values is/are reached.

The SCPU device, in the second level environmental monitoring system, will also perform (a) at least an overall assessment and/or (b) at least an overall forecast related to the health concern of the occupants and/or safety concern of the environment based on data and/or all information obtained from the LCPU device and/or the values of the external parameter from the external database(s). The overall assessment and/or the overall forecast on a particular group of interested will be concluded instantly based on the classification of the fundamental data according to a forth judgment principle.

In the present invention, the environmental monitoring device and system further comprise a plurality of sensor modules of same or different types of health parameter sensors for being clipped to the clothing, being carried around by the occupant, and/or being put in any location as desired by the occupants temporarily or permanently to obtain and the health related data of the occupant continuously at specific pre-set time period.

The health related data of the occupant obtained by the sensor modules is further sent to the LCPU device and further to the SCPU device through the LCPU device. Each sensor module has its own unique identity to match further with the health related data of the occupant. The health related data, being undergone a first level of data processing by the LCPU device, to generate some summarized information. The summarized information is then sent to the SCPU device. The summarized information of them includes at least one of the following: an average, a median, mean of the obtained value, the increasing/decreasing rate of the obtained values, and the frequency and duration of when the alerted values (the values exceed curtained pre-defined level) are recorded.

In the an example, in a region where the air is filled with particulate matters that up to a concentration could lead to allergic disease to certain group of occupants, such as certain level of PM10 or PM2.5, that can induce to chronic cough, the sensor modules containing dust sensors will record the profiles and levels of the particulate matters (e.g. PM10 or PM2.5) by occupants within a region, this information is send to the SCPU through the LCPU device. The sensor modules containing noise receivers or a microphones record the frequencies, intensities and/or profiles of the cough patterns (as one of the health related parameters) by the occupants in the same region, these information will also send to the SCPU through the LCPU device. The personalized data by the occupants may contain information as different occupations and different ages, which allows the occupants to be classified according to different residing environment and different age group. The analysis on the following may be built:

(a) A type correlationship analysis on the amount of particulate matters (e.g. PM10 or PM2.5) maybe inhaled by the occupant in different residing environment; and
(b) A numerical correlationship analysis on the amount of particulate matters (e.g. PM10 or PM2.5) maybe inhaled by the occupant of different ages group.

The environmental and health correlationship analysis is further performed by the SCPU, on the obtained values of environmental parameter(s) and the health related data of the occupant in relations with the personalized data. The result is sent from the SCPU devices to the LCPU device and is employed to update the defined alerted level in any analysis.

For instance, in the first example, the analysis on the following may be further built:

(1) The concentration of particulate matters (e.g. PM10 or PM2.5) in relation to chronic cough against different age's group is built; and
(2) The concentration of particulate matters (e.g. PM10 or PM2.5) in relation to chronic cough against different residing environment is built.

In one embodiment, the environmental monitoring system the LCPU and SCPU comprise separate power supplies to support the operation.

The following figures and description reveal the further details of the present invention.

### BRIEF DESCRIPTION OF THE DRAWINGS

FIG. 1 illustrates circuit modules of the environmental device of the present invention;

FIG. 2 depicts a block circuit diagram of the environmental device of the present invention;

FIG. 3 depicts a circuit diagram for temperature sensor in the environmental device of the present invention;

FIG. 4 depicts a circuit diagram for relative humidity sensor in the environmental device of the present invention;

FIG. 5 depicts a circuit diagram for volatile organic compounds sensor in the environmental device of the present invention;

FIG. 6 depicts a circuit diagram for carbon monoxide sensor in the environmental device of the present invention;

FIG. 7 depicts a circuit diagram for carbon dioxide sensor in the environmental device of the present invention;

FIG. 8 depicts a circuit diagram for dust sensor in the environmental device of the present invention;

FIGS. 9 to 13 depict examples of the parameter judgment standards and criteria, as well as the resulted implications; and

FIG. 14 depicts a flowchart of the environmental monitoring and analyzing by the present invention.

FIG. 15 depicts a block circuit diagram of the environmental device of the present invention where the input/output port is can be communicate with another computer outside the device.

FIG. 16, the depicts a block circuit diagram of the environmental device of the present invention; wherein the device is a part of the component which is being included in any unit and modules of the air equipment

### DETAILED DESCRIPTION OF THE DRAWINGS

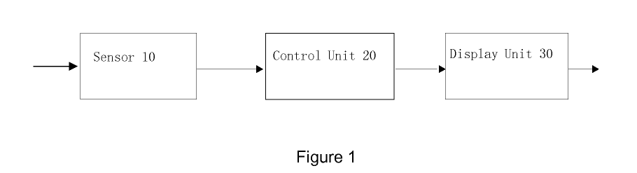

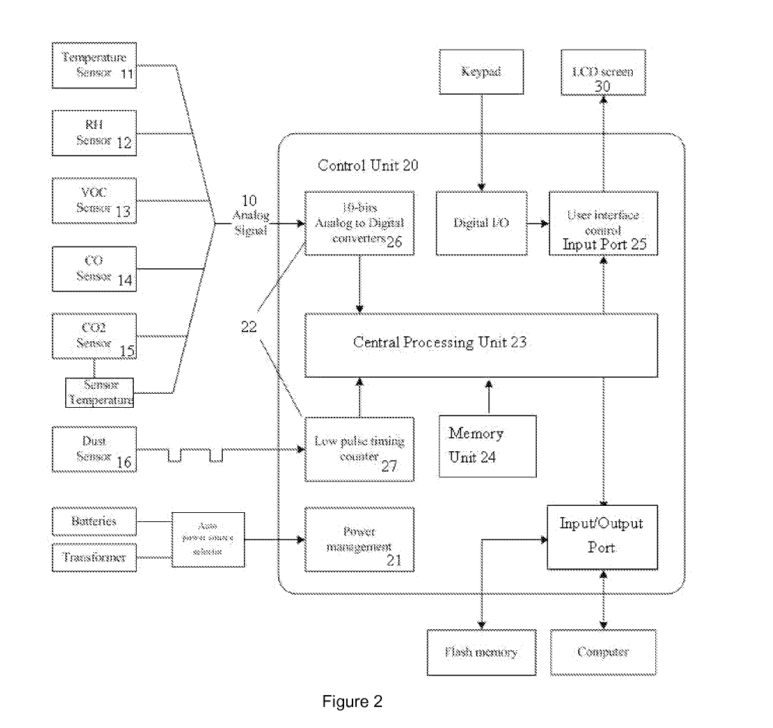

Referring to FIGS. 1 and 2, the device of the present invention contains the sensors 10, the control unit 20 and the display unit 30.

The sensors 10 obtain the values of different environmental parameters. The control unit 20 collects the obtained values. In the present embodiment, the sensors 10 are a temperature sensor, a relative humidity sensor 12, a volatile organic compounds sensor 13, a carbon monoxide sensor 14, a carbon dioxide sensor 15, and a respirable suspended particulates sensor 16. Other environmental sensors such as the ozone sensor, the nitrogen dioxide sensor, the air flow rate sensor, the radon level sensor and the formaldehyde sensor can be applied for the same purpose.

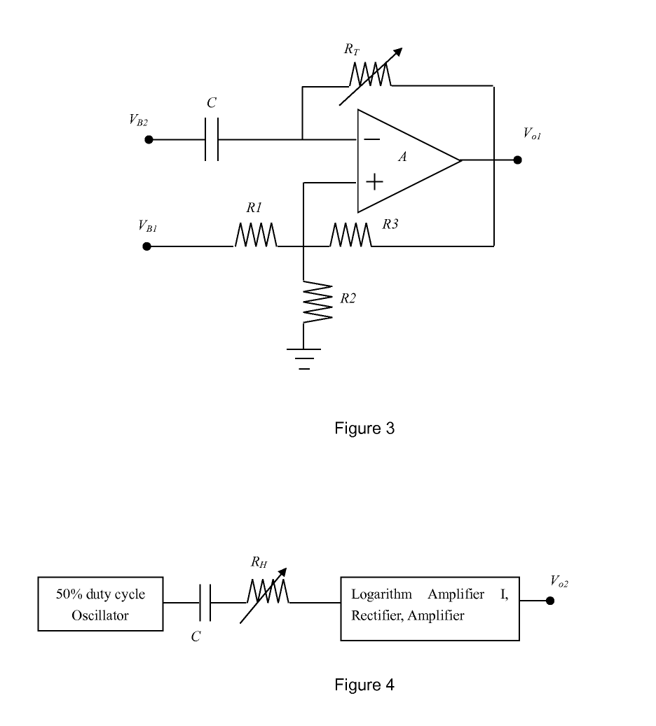

FIGS. 3-8 indicate the circuit diagrams for the sensors in the embodiment of the present invention. The circuit for the temperature sensor 11 is shown in FIG. 3. In the present embodiment, a thermistor in which its resistance varies with the temperature is employed as the temperature sensor. The change of temperature in the environment results the change of the resistance of the thermistor R_{T}. The change of thermistor R_{T} can be represented by the voltage output. The control unit 20 receives the output voltage Vol. The output of the temperature sensor belongs to a chain of periodic signals, whereas the frequencies of the periodic signals are temperature dependent. The control unit 20 detects the frequency of the waveform and determines the measured temperature.

FIG. 4 indicates the circuit for the relative humidity sensor 12. In the present embodiment, the relative humidity sensor 12 belongs to a resistive type relative humidity sensor. A capacitor C is connected in series to a humidity sensitive resistor R_{H}. The circuit amplifies and blocks out all DC component of the signals obtained from the sensor. The signal is output as voltage. The circuit is effective to block off the entire DC component and protect the humidity sensitive resistor R_{H}. It is a simple circuit and adaptive to different duty cycles of the input signals. In the present embodiment, a 50% oscillation duty cycle is employed.

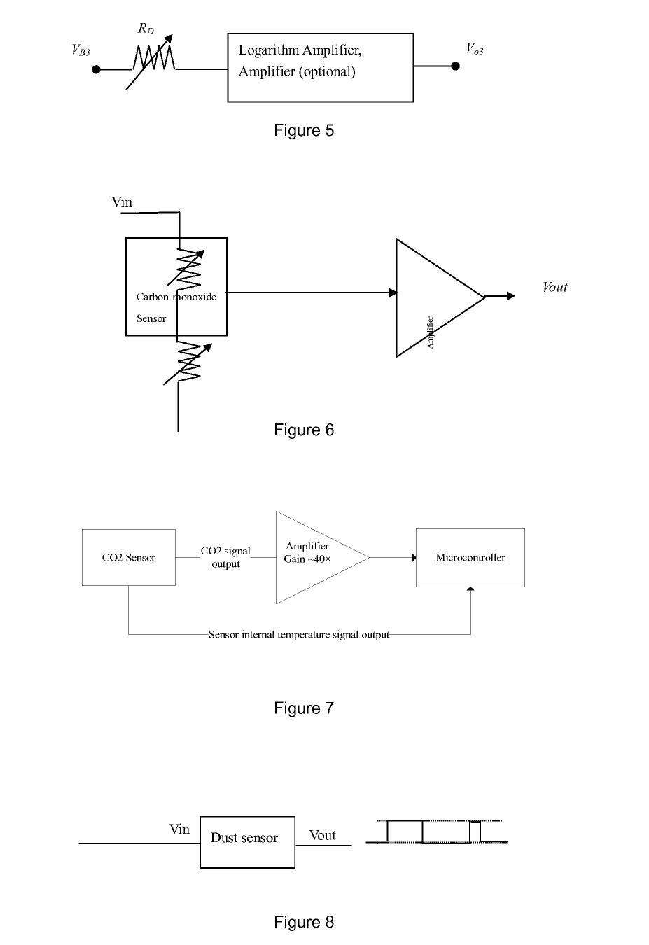

FIG. 5 indicates the circuit for the sensor of volatile organic compounds 13. In the present embodiment, the sensor of volatile organic compounds 13 belongs to a heated metal oxides type. The sensor varies its resistance R_{D} with the concentration of volatile organic compounds. The input voltage V_{B3} would first go through the resistor with resistance R_{D}, it will then be amplified by an analog amplifier. The voltage output is then sent to the control unit.

FIG. 6 indicates the circuit for the carbon monoxide sensor 14. In the present embodiment, the carbon monoxide sensor 14 being employed belongs to a heated metal oxide type sensor. The sensor varies its resistance with the concentration of carbon monoxide. The input voltage would first go through the resistor, it will then be amplified by an analog amplifier. The voltage output is then sent to the control unit.

FIG. 7 indicates the circuit of carbon dioxide sensor 15. In the present embodiment, the carbon dioxide sensor 15 belongs to a heated metal oxide type. A heating element is included in addition to the sensor element. The resistance of the sensor changes with the concentration of carbon dioxide. The input voltage first go through the resistor, it will then be amplified by an analog amplifier and be sent to the control unit 20. In order to obtain an accurate value for carbon dioxide, the desired operation temperature of the sensor is maintained by the built-in heater. The influence of the environmental temperature and ambient carbon dioxide is eliminated by comparing the voltage output obtained with that of the ambient air. A more accurate result is obtained. In addition, the internal temperature of the sensor by the heating element is fed to control unit 20. This acts as a reference for showing that the sensor has been warmed-up, and indicating that sensor has reached the optimal operation temperature.

FIG. 8 indicates the circuit for the dust sensor 16 in the present embodiment. In the present embodiment, the dust sensor 16 belongs to a light scattering type sensor. The output of dust sensor will go to low voltage (ground level) when the particulate matters are detected, otherwise the output will stay at high voltage. In other words, the low pulse occupancy time is proportional to dust concentration. By obtaining the ratio of the time of total low pulse and total high pulse, the control unit 20 would be able to calculate the corresponding dust level.

The control unit 20 in the present embodiment comprises a power supply and control circuit 21, a voltage input circuit 22, a central processing unit 23, a memory unit 24 and a voltage output circuit 25. The power supply and control circuit 21 connect an external power supply to the device. The external power supply could be either AC or DC power supply. When inserting a power plug to the present embodiment, the auto power source selector directs the power source to transformer.

The voltage input circuit 22 collects the values obtained from the sensors 10. In the present embodiment, the voltage input circuit 22 includes an analog to digital converter 26 and a low pulse time counter 27. The analog to digital converter 26 receives the analogue signals from the temperature sensor 11, the relative humidity sensor 12, the volatile organic compounds sensor 13, the carbon monoxide sensor 14, and the carbon dioxide sensor 15, as well as the reference signals by the carbon dioxide sensor 15. The analog to digital converter 26 converts the analogue signals to digital signals, and inputs the digital signal into the central processing unit 23. The low pulse time counter 27 obtains the input signal from the dust sensor circuit. The central processing unit 23 collects an average value of low pulse timing from dust sensor circuit. The types of sensors employed determine the voltage input circuit. The voltage input circuit can be modified to fit with different sensors types.

The memory unit 24 stores the first judgment principle, the second judgment principle and the third judgment principle, as well as the user-friendly interpretation of the obtained values based on the parameter ranges and a recommendation in response to the obtained values based on the parameter ranges that is easily understood by a non-technical user;

The first judgment principle defines at least two-parameter ranges for each environmental parameter. The values of environmental parameter refer to the values obtained by the sensors 10, such as the values obtained by the temperature sensor, the relative humidity sensor, the volatile organic compounds sensor, the carbon monoxide sensor, the carbon dioxide sensor and the dust sensor in the present embodiment. For example, the parameter ranges for the temperature could be referred to the ranges of “>25.5° C.”, “<20° C.” and “<10° C.” etc. The second judgment principle defines at least one the conditional arrays, the at least two parameter ranges defined by the first judgment principle for use as the parameter ranges for defining each conditional array. For example, the parameter range for the temperature in an occasion is defined as “25.5-35° C.” and the parameter range for the volatile organic compounds in the same occasion is defined as “>600 μg/m³”. A parameter range defined by the first judgment principle can applied for defining different conditional arrays. Air-quality-level judgment standards for air quality levels are defined based on the combination of different categories of the measured environmental parameters

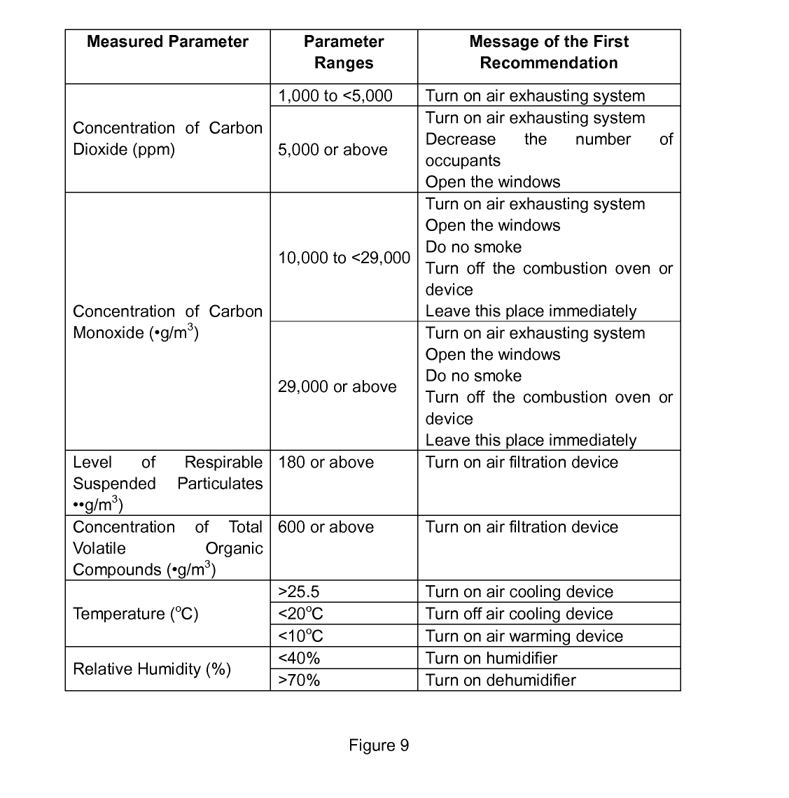

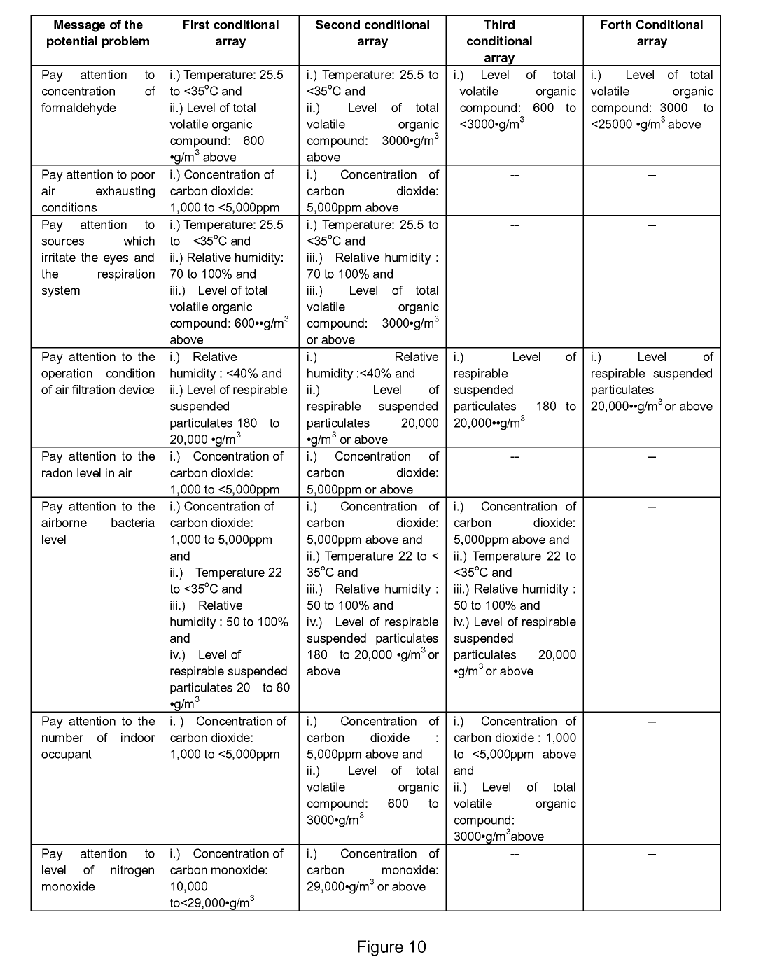

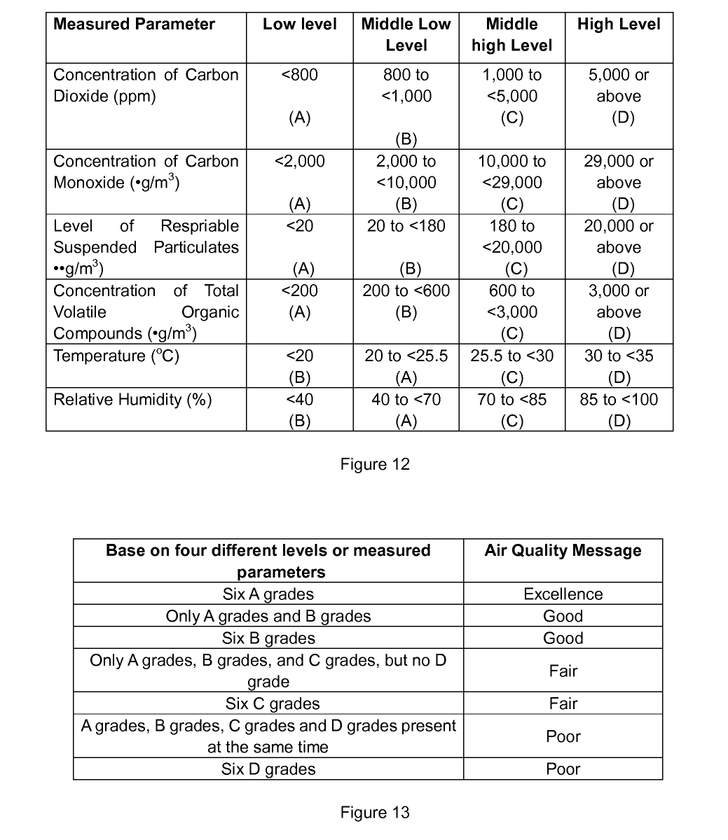

The messages provided include the message corresponding to the potential problems based on the parameter ranges, the recommendations to address the potential problems and the message corresponding to the air quality level. For example, as indicated in FIG. 9, when the parameter range of temperature is defined as “>25.5° C.”, the recommendation in response to the obtained values based on the parameter range is “Turn on air cooling devices”. A message corresponding to potential problems for each conditional array is provided, based on the second judgment principle. Referring to FIG. 10, for example, when the temperature is in the parameter range of “25.5-35° C.” and the level of the total volatile organic compounds is in the parameter range of “above 600 μg/m³”, the message corresponding to the potential problem for this conditional array is “high level of formaldehyde”. The recommendations to address the potential problem comprise “Open the windows”, “Turn on air filtration device”, “Turn on air exhausting system” and “Do not smoke”. FIGS. 12 and 13 indicate the air quality level, which is defined by the air-quality-level judgment standards based on the third judgment principle.

Further refer to the FIG. 10, the first conditional array showing the environmental parameters of temperature and total volatile organic compound are employed for assessment of the level of formaldehyde. When the temperature is within the range of 25.5° C. to <35° C. (which is the optimal range for emission of the formaldehyde), and when the level of total volatile organic compound is 600 ••g/m³ above, the formaldehyde level is forecasted to be a problematic and message of this potential problem will be displayed. However, when the level of total volatile organic compound is in the range of 3000 to <25000 •g/m³, the reading from the temperature will become ignored in the assessment and forecast of the level of the formaldehyde. This is because the level of the total volatile organic compound is already become a dominant factor in the assessment and the forecasting of the level of formaldehyde. In indoor environment where the concentration of total volatile organic compound is in the range of 3000 to <25000 •g/m³, the concentration of formaldehyde is always in an alert level. In this case, the first conditional array is automatically shifted to the forth conditional array. The temperature sensor will be turned off automatically in the environmental monitoring device for power saving. When the concentration of total volatile organic compound drop back to the level of just above 600 •g/m³, the environmental parameter of the temperature will be re-considered again, and the forth conditional array is automatically shifted another pre-defined conditional array.

The central processing unit 23 receives the signals from the voltage input circuit 22. The voltage input circuit 22 converts all analogue signals from the sensor circuit 20 into digital signals.

The digital signals are then judged against with the predetermined standards and criteria, which are stored in the memory unit 24 under the first judgment principle defining and obtaining the parameter range. Recommendations are provided.

The obtained values are also judged against with the predetermined standards and criteria which are stored in the memory unit 24 under the second judgment principle. The second judgment principle defines the conditional arrays. At least two parameter ranges defined by the first judgment principle for use as the parameter ranges for defining each conditional array. Based on the interrelationship of the obtained values of the different environmental parameters, a message corresponding to the potential problem for the conditional array and recommendations to address the potential problems are provided.

The obtained values are also judged against with the predetermined standards and criteria which are stored in the memory unit 24 under the third judgment principle. The air-quality-level judgment standards for air quality level are defined based on the combination of different categories of the measured environmental parameters. A message corresponding to air quality level by the air-quality-level judgment standards is provided. The display unit 30 output the individual measured values and the messages by the voltage output circuit 25. The displays are in any formats, wordings, numerical, and graphical characters.

The device of the present invention contains input ports and input/output ports, whereas the input ports receive input signal from the keypad. The input/output ports transfer the information to other devices, such as computer, pocket size personal computer and flash memory. The input/output ports connect the device to other devices by an infra-red interface device, Bluetooth interface device and other wireless interface devices.

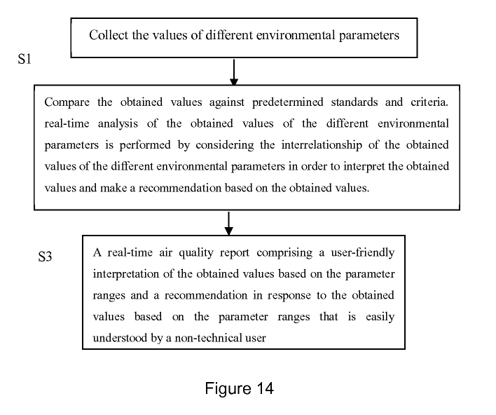

FIG. 14 indicates the method of environmental monitoring and analyzing by the present invention. The sensors S1 obtain values of different environmental parameters. The values are then sent to the control unit. The control unit in S2 compares the obtained values of the environmental parameters against the predetermined standards and criteria. Based on the interrelationship of the obtained values of the different environmental parameters, real-time analysis of the obtained values of the different environmental parameters is performed. A user-friendly interpretation of the obtained values based on the parameter ranges and recommendations in response to the obtained values based on the parameter ranges are output and displayed in the display unit S3. The first judgment principle defines the parameter ranges for each measured environmental parameter. The second judgment principle defines the conditional arrays. At least two parameter ranges defined by the first judgment principle are employed the parameter ranges for defining each conditional array. The third judgment principle defines the categories for each measured environmental parameter. An overall air quality level is defined by the air-quality-level judgment standards based on the combination of different categories of the measured environmental parameters. A message corresponding to air quality level by the air-quality-level judgment standards is provided.

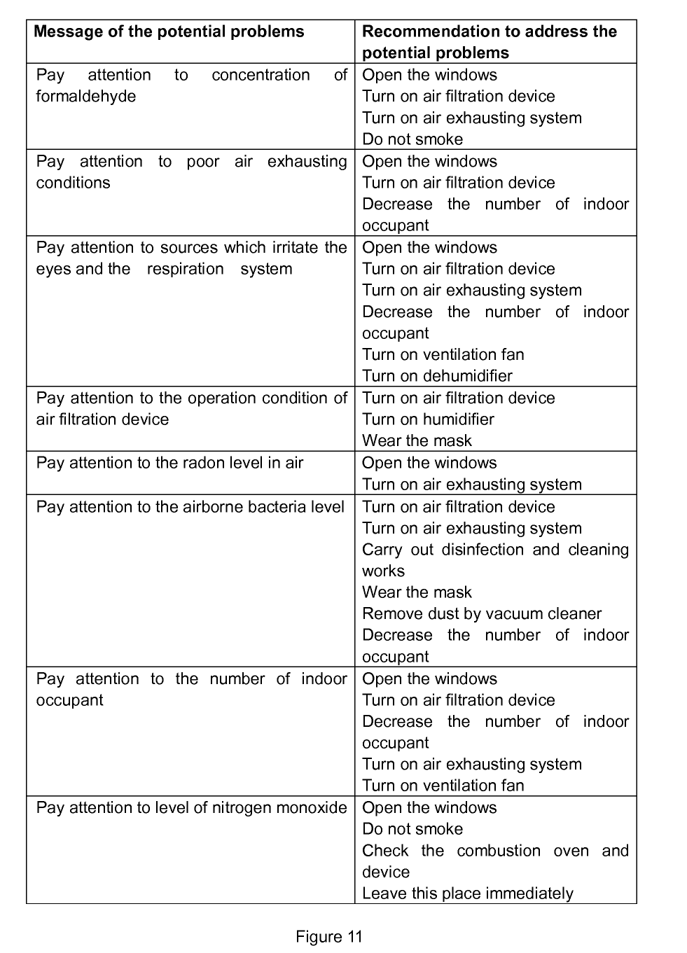

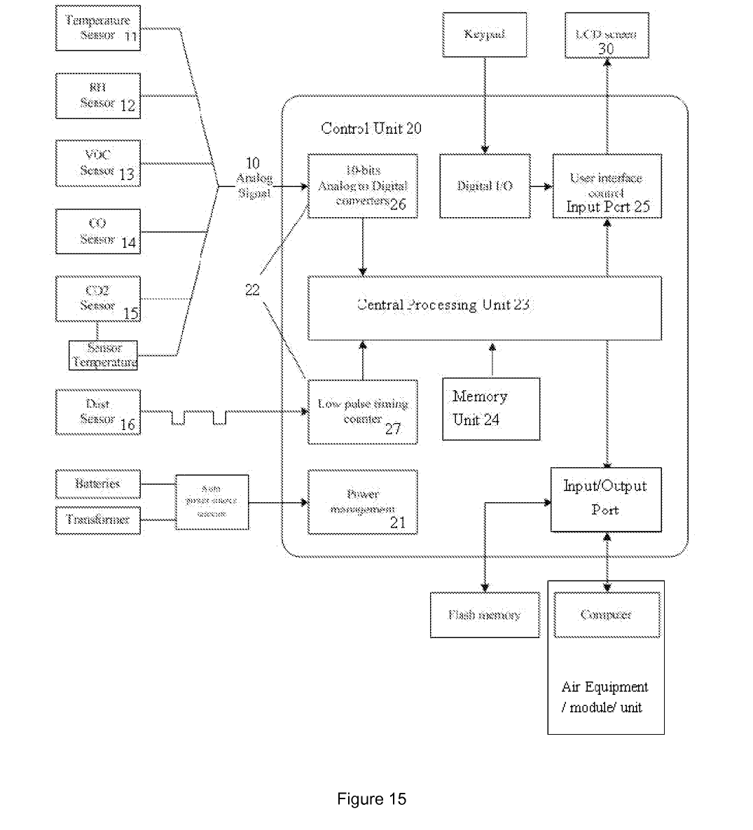

Further refer to FIG. 2 and FIG. 15, the input/output port is can be communicate with another computer outside the device. In one embodiment, the said another computer is being possessed by an air treatment unit. The central processing units of the said air treatment unit receive the messages corresponding to the said real-time air quality report from the device; and based on the message to establish setting and parameter values for the operating condition of the said air treatment unit. In such case, the corresponding air treatment unit is instructed to be operated at appropriate settings or parameter values, for improving and mitigating the problematic environmental parameters accordingly, and or for prevent the forecasted problematic condition to be happened. For example, when the sensors of temperature, relative humidity, carbon dioxide and respirable suspended particulates are used for forecasting the level of the airborne bacteria level (refer to FIG. 10), and when the level of forecast is high and up to a level that the turning on the air filtration device is required (refer to FIG. 11). A message regarding this will be sent to the central processing unit of the air filtrating device directly. The central processing unit of air filtrating device will automatically instruct the air filtration device to operate at appropriate operating condition.

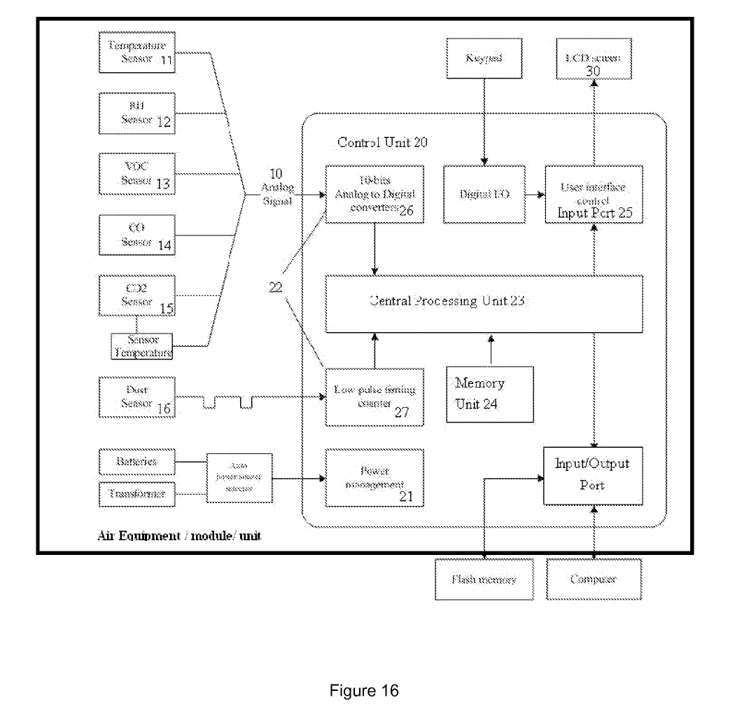

Refer to FIG. 16, the device according to claim 1, wherein the device is a part of the component which is being included in any unit and modules of the air equipment containing one or the combination of the components from: fan of any type, blower, pump, drawer, filtration apparatus and/or filter for air pollutants of any type, apparatus for sterilizing the air, apparatus for environmental humidity controlling, apparatus for the environmental temperature controlling, apparatus for environmental air flow controlling, apparatus for controlling environmental brightness. In another words, the device is being possessed by the air equipment. The control unit of the device establishes the setting and the parameter values for the operating condition of the air equipment based on the obtained values of the environmental parameters and/or the simultaneous forecast and instant level assessment of at least one environmental parameter not obtained by the plurality of sensors.

What is claimed is:

1. An environmental monitoring device, comprising:
a plurality of sensors of different types, where each different type of sensor obtains values of different environmental parameters; and
a control unit that performs real-time analysis of different environmental parameters by considering the interrelationship of the obtained values of the different environmental parameters and provides an instant level assessment of at least one environmental parameter not obtained by the plurality of sensors.

2. The environmental monitoring device according to claim 1, wherein the control unit further comprises a central processing unit that stores predetermined standards and criteria.

3. The environmental monitoring device according to claim 1 further comprising a display unit that displays a real-time air quality report.

4. The environmental monitoring device according to claim 3, wherein the real-time air quality report further comprises at least one from the group consisting of:
(a) a recommendation based on the obtained values;
(b) a simultaneous forecast that provides an instant level assessment of at least one environmental parameter not obtained by the plurality of sensors and further including an instant level assessment of at least one selected from the group consisting of: formaldehyde, airborne bacteria, radon and nitrogen monoxide, carbon dioxide, carbon monoxide, respirable suspended particulates, ozone, air flow rate, fungi level, total volatile organic compounds, temperature, relative humidity, dew point, air pressure, wind speed, and overall air quality;
(c) the potential health problem to an occupant caused environment;
(d) health risk assessment of an occupant;
(e) a recommendation on improving the environment;
(f) an improved health condition of the occupant due to the environment;
(g) health effects of the occupant due to the environment;
(h) sources which irritate the eyes and the respiration system;
(i) at least one of the detected number and the recommended number of indoor occupants;
(j) at least one of the current conditions and recommended operation conditions of one of air exhausting conditions, air ventilation conditions, and operation condition of an air filtration device;
(k) a comment about the health effect on the short term and the long term exposure by the environmental parameters;
(l) a recommendation on the operation of at least one of the following equipment consisting of: an air exhausting system, a humidifier, a dehumidifier, an air warming device, an air cooling device, an air filtration device, a combustion device, a ventilation fan, and a vacuum cleaner;
(m) a recommendation on at least one of human action from the group consisting of: opening the window, decreasing the number of occupants, leaving the place immediately, not smoking, wearing a mask, carrying out disinfection and cleaning works, and removing dust.

5. The environmental monitoring device according to claim 1, wherein the environmental monitoring device further is linked to at least one device from a group consisting of: an environmental monitoring station, databases, an information center, and a system regarding the pollutant levels and/or the pollution index of the regional outdoor air quality.

6. The environmental monitoring device according to claim 1, further comprising a plurality of health parameter sensors of different types to obtain values of different health parameters for at least one occupant and where the environmental monitoring device further connects to at least one health parameter sensor by means of at least one of a wired and a wireless data communication and where the environmental monitoring device further performs real-time analysis by considering an interrelationship of the obtained values of the different environmental parameters and the data obtained by a health parameter sensor and/or monitor.

7. The environmental monitoring device according to claim 1, further comprising input/output ports for transferring information to other devices through an infra-red interface device, or a Bluetooth interface device or other wireless interface devices.

8. The environmental monitoring device according to claim 1, wherein a cloud storage system is simultaneously linked to multiple units of environmental monitoring devices through at least one of a wired or a wireless data transmission method.

9. The environmental monitoring device according to claim 1, wherein the values of the environmental parameters obtained by the plurality of sensors from multiple units of environmental monitor device are dynamically uploaded and co-stored to a cloud storage system.

10. The environmental monitoring device according to claim 1, wherein the real-time analysis of different environmental parameters is performed in a cloud computing system and predetermined standard and criteria are stored in the memory in the cloud storage system where the stored predetermined standard and criteria are extracted from the cloud storage system by the cloud computing system to perform the real-time analysis of different environmental parameters.

11. The environmental monitoring device according to claim 1, wherein the real time analysis is performed on the environmental parameters obtained from the plurality sensors of multiple units of environmental monitoring device.

12. The environmental monitoring device according to claim 1, wherein at least one of a cloud storage system and a cloud computing system is further linked by a wired or wireless data transmission method to at least one environmental device when the at least one environmental device is operating and is not operating and the values of different environmental parameters obtained by the plurality of sensors are different.

13. The environmental monitoring device according to claim 1, wherein the plurality of sensors and the control unit are powered by different power sources and the values of the different environmental parameters obtained by the plurality of sensors are transmitted to the control unit through at least one of a wired and a wireless data transmission method.

14. The environmental monitoring device according to claim 1, wherein the control unit and the central processing unit are powered by different power sources and the values of the different environmental parameters obtained by the plurality of sensors are transmitted to the control unit through at least one of a wired or a wireless data transmission method.

15. The environmental monitoring device according to claim 1, wherein the control unit and the display unit are powered by different power sources and the control unit transmits the real-time air quality report to the display unit for display through at least one of a wired a wireless data transmission method.

16. The environmental monitoring device according to claim 1, wherein each individual sensor or several sensors from the plurality of sensors are powered by different power sources.

17. The environmental monitoring device according to claim 1, wherein the control unit is further linked by at least one of a wired or a wireless data transmission method to at least one power control, where the at least one power control supplies the power to operate different environmental devices and when an environmental device is in one of an operating state and a non-operating state, the values of different environmental parameters obtained by the plurality of sensors are different.

18. The environmental monitoring device according to claim 1, wherein the control unit is further linked by at least one of a wired and a wireless data transmission method to at least one environmental device where when the at least one environmental device is in an operating state or an non-operating state, the values of different environmental parameters obtained by the plurality of sensors will be different.
